(19) United States
(12) Patent Application Publication
(10) Pub. No.: US 2010/0274254 A1
(43) Pub. Date: Oct. 28, 2010

(54) SURGICAL INSTRUMENTATION FOR FITTING AN OSTEOSYNTHESIS NAIL

(76) Inventors: Pascal Boileau, Nice (FR); Philippe Clavert, Illkirch (FR)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/428,352

(22) Filed: Apr. 22, 2009

(51) Int. Cl. A61B 17/58; A61B 17/56

(52) U.S. Cl. 606/93; 606/62; 606/96

(57) ABSTRACT

Embodiments of a method for introducing a substance near a fracture of a bone include installing an osteosynthesis nail into a canal of the bone, the osteosynthesis nail including a fixation aperture, coupling an alignment device with the osteosynthesis nail, the alignment device including an alignment sleeve having a longitudinal axis passing through the fixation aperture, drilling a hole through a wall of the bone and into the canal using the alignment sleeve, inserting an injector through the alignment sleeve, the injector having an injector body with an injection nozzle and a plunger, and moving the plunger to push the substance from within the injector body through the injection nozzle and into the canal.

### TECHNICAL FIELD

Embodiments of the present invention relate generally to surgical instrumentation for fitting an osteosynthesis nail, and more specifically to systems and methods for treating a bone fracture associated with such instrumentation.

### BACKGROUND

When a bone such as the humerus, the femur, or the tibia is broken, an osteosynthesis nail may be fit lengthwise inside the medullary canal of the bone. This nail stabilizes the bone fragments surrounding the fracture which is to be treated. In order to minimize incisions into the soft tissue around the bone fracture site, the nail is often inserted into the bone via the bony end of the medullary canal, after first making an opening in the medullary canal. An alignment device with guide holes may then be used to secure, at several points along the nail, a number of transverse fixing screws between the nail and the bone. Each of these fixing screws may then be engaged percutaneously in a mating passage defined through the nail, the correct relative positioning of the guide hole with respect to the passage in the nail achieved by the mechanical relationship between the alignment device and the nail.

Current systems for introducing biochemical treatment liquids at or near the fracture site often involve inserting an injector body lengthwise into the medullary canal prior to installation of the osteosynthesis nail; however, such systems often take a long time to deploy, and the subsequent installation of the osteosynthesis nail can reposition the bone fragments around the nail and/or displace the biochemical treatment liquids, thereby preventing the liquids from effectively treating the fracture. Other systems often involve using an osteosynthesis nail with special or customized features, such as grooving the outer face of the nail along which the treatment liquid travels, or using the interior of a screw as a canula for carrying the fluid. Such systems often require customized and expensive osteosynthesis equipment, and often cannot be used with existing osteosynthesis nail systems.

### SUMMARY

Embodiments of the present invention include surgical instrumentation which not only allows a pre-existing osteosynthesis nail to be fitted in a reliable and relatively uninvasive way, but also allows the easy and quick injection of a liquid as close as possible to the site of the bone fracture. Embodiments of the present invention make use of an alignment device, currently used for fitting transverse fixation screws between the osteosynthesis nail and the bone, for the additional purpose of injecting biochemical treatment liquid percutaneously, while identifying reliably and precisely the injection zone to ensure that it coincides as closely as possible with the bone fracture site to be treated. According to embodiments of the present invention, the fitting of the injector body is thus no more invasive than the fitting of the transverse fixation screws. Furthermore, the liquid is injected when the nail is already housed in the medullary canal of the bone, which does not significantly lengthen the duration of the surgical operation and which ensures that the liquid acts on the bone fragments stabilized by the nail, according to embodiments of the present invention. Embodiments of the present invention thus remarkably improve the quality of the bone reconstruction at the fracture site.

Embodiments of a surgical instrumentation (1) for fitting an osteosynthesis nail (2) according to embodiments of the present invention include an alignment device (10) suitable for positioning, extracutaneously and relative to the nail when the nail is housed in the medullary canal (32) of a bone (3), at least one sleeve (14) for guiding a tool for the percutaneous preparation of the bone, such as a percutaneous drill bit, for fitting a transverse fixation element, such as a screw, between the bone and the nail, characterized in that the instrumentation also comprises a system (20) for the percutaneous injection of a liquid (5), which comprises on the one hand an injector body (21) suitable to be inserted in an essentially mating manner into the guide sleeve (14), until an end part (23) of this injector body passes transversely through the wall (31) of the bone (3) and into the medullary canal (32) of this bone while the nail (2) is housed in this medullary canal, and on the other hand a plunger (22) for pushing the liquid contained in the injector body (21) through its end part (23).

According to some embodiments of the present invention, the end part (23) of the injector body (21) has dimensions such that, when the nail (2) is housed in the medullary canal (32) of the bone (3), it can enter a transverse fixation aperture (2₁ to 2₄). According to other embodiments of the present invention, the end part (23) of the injector body (21) has dimensions such that, when the nail (2) is housed in the medullary canal (3₂) of the bone (3), the end part (23) can lie within all or part of the transverse fixation aperture (2₁ to 2₄). According to some embodiments of the present invention, the end part (23) of the injector body (21) is provided, at its free end, with an orifice (24) for the liquid to flow out of the injector body, the outflow orifice being positioned essentially coaxially with the guide sleeve (14) when the injector body is inserted into the guide sleeve. According to some embodiments of the present invention, the end part (23) of the injector body (21) is provided, in its long region, with at least one orifice (25) for the liquid (5) to flow out of the injector body, this outflow orifice being centered on an axis perpendicular to the central axis (Z-Z) of the guide sleeve (14) when the injector body is inserted into the guide sleeve.

A surgical kit for treating a fracture (F) of a bone (3) according to embodiments of the present invention includes instrumentation (1) as described above, an osteosynthesis nail (2) suitable for being fitted by the instrumentation (1) into the medullary canal (3₂) of the bone (3), its positioning being linked to that of the alignment device (10) of this instrumentation, and a liquid (5) for treating the fracture (F), injectable by the percutaneous injection system (20) of the instrumentation (1) into the medullary canal (3₂) of the bone (3). The osteosynthesis nail may be a humeral, femoral, or tibial nail, according to embodiments of the present invention. The liquid (5) may be a fluid solution containing biochemical substances such as, for example, growth factors. The liquid (5) may be a viscous solution lending mechanical reinforcement to the fracture such as, for example, a biological cement.

A surgical method for treating a fracture of a bone according to embodiments of the present invention includes installing the nail in the medullary canal of the bone by inserting it into the canal via one of the ends of the bone, positioning a guide sleeve extracutaneously and relative to the nail, inserting percutaneously a bone preparation tool such as a percutaneous drill bit through the guide sleeve until an opening is made passing transversely through the wall of the bone into the medullary canal, inserting a substantially mating injector body through the guide sleeve until an end part of the injector body passes through the hole made with the aid of the percutaneous preparation tool and thus enters the medullary canal of the bone, pushing a liquid contained in the injector body through its end part so that the liquid is spread between the nail and the bone wall, and after withdrawing the injector body, inserting a transverse fixing element such as a screw through the guide sleeve, between the bone and the nail, until the fixing element lies inside a dedicated fixation passage defined through the nail.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a front perspective view of a treatment kit including an osteosynthesis nail, an alignment device, an alignment sleeve, and an injector, shown in relation to a partial view of a bone canal, according to embodiments of the present invention; and

FIG. 2 illustrates a side partial cross-sectional view of the alignment sleeve, bone canal, and osteosynthesis nail of FIG. 1, taken along plane II of FIG. 1, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

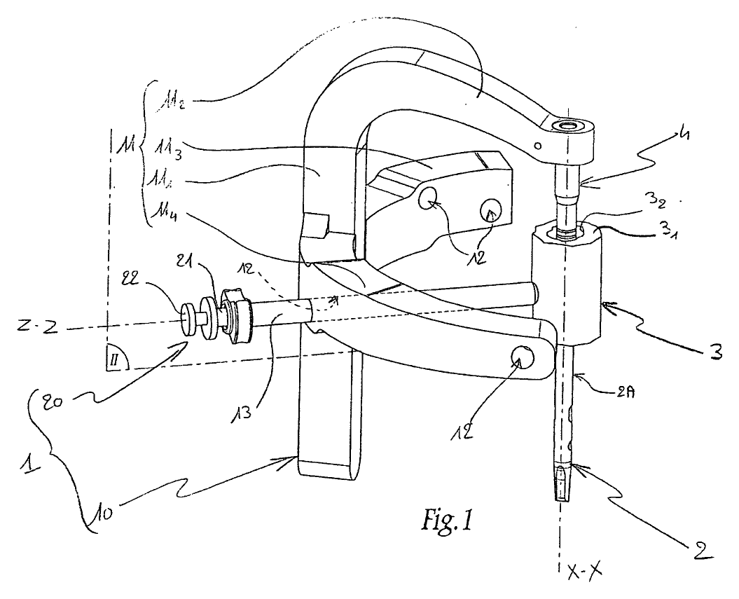

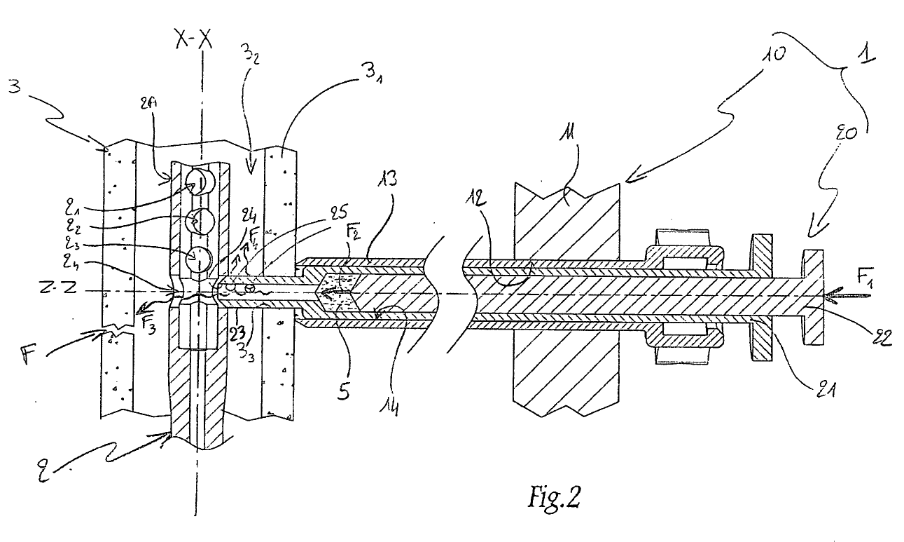

FIGS. 1 and 2 show surgical instrumentation 1 designed for fitting an osteosynthesis nail 2 in a bone 3, according to embodiments of the present invention. In the figures, the bone 3 is shown partially and diagrammatically, essentially to indicate the generally tubular shape of the bone, with its bone wall marked 3₁, while its open internal volume corresponding to the medullary canal of the bone is marked 3₂. As an example, the bone 3 may be a long bone such as a humerus, a femur, or a tibia, so that in the anatomical position of these three examples of bones, the overall centerline of the medullary canal 3₂ is on an approximately vertical axis, as shown in FIGS. 1 and 2.

The osteosynthesis nail 2 may be similar or the same as an existing osteosynthesis nail known to one of ordinary skill in the art, based on the disclosure herein. Osteosynthesis nail 2 has an approximately cylindrical outer face 2A with a circular base centered on an axis X-X. Fitting or installing the nail 2 involves positioning the nail 2 inside the bone 3 so that it lies lengthwise inside the medullary canal 3₂, in a generally coaxial manner, as shown diagrammatically in FIGS. 1 and 2.

The nail 2 contains a number of through passages extending radially relative to the axis X-X. Four through passages are illustrated in the part of the nail 2 shown in FIG. 2, to which the respective references 2₁ to 2₄ are given. These passages are distributed along the longitudinal direction of the nail 2 and their centerlines lie on respective axes that are oriented in different directions in a plane perpendicular to the axis X-X. Each of these passages 2₁ to 2₄ is designed to take a screw or similar fixing element (not shown), so that the nail 2 and the bone 3 can be fixed transversely to each other.

The instrumentation 1 comprises an alignment device 10 designed to identify, extracutaneously, the positions of the passages 2₁ to 2₄ of the nail 2 when the nail 2 is housed inside the medullary canal 3₂ of the bone 3. For this purpose, the alignment device 10 includes a body 11 with an elongated straight main part 11₁ designed, in use, to lie roughly parallel to the bone 3 but outside of the soft tissue surrounding the bone. In the example of a humeral nail, this part 11₁ of the body 11 thus lies along the arm of a patient, while the soft tissue of the arm lies between the part 11₁ and the humerus.

At one of its longitudinal ends, the part 11₁ of the body is extended by a curved part 11₂ whose dimensions are such that, when in use, part 11₂ fits over the end of the bone 3 where the opening in the medullary canal 3₂ has been made. At its free end, this part 11₂ of the body carries a screw 4 providing mechanical connection to the nail 2. When the nail 2 is assembled onto the body 11 by this screw 4, the angular position of the nail 2 about its axis X-X is set in a predetermined manner.

In its long region, the part 11₁ of the body is provided with two lateral projecting parts 11₃ and 11₄, each forming an arc extending orthoradially around the bone 3. As such, in a cutting plane at right angles to the part 11₁ of the body passing through the parts 11₃ and 11₄, the general profile of the body 11 is a C.

As shown in FIG. 1, each of the parts 11₃ and 11₄ has through holes 12, whose centerlines lie on respective axes Z-Z which, when the nail 2 is fixed to the body 11 by the connecting screw 4, extend radially with respect to the axis X-X of the nail 2, and are each aligned with one of the passages 2₁ to 2₄ of the nail. Each of these through holes 12 is designed to be fitted internally with a tubular sleeve 13, the centerline of whose transverse section lies on the axis Z-Z and which is adjusted externally to the internal transverse section of the hole 12. FIG. 1 shows only one of these sleeves 13.

In practice, each sleeve 13 may fit in a sliding manner in the corresponding hole 12, while optional features (not shown) allow the position of the sleeve 13 to be reversibly locked with respect to the body 11. According to some embodiments of the present invention, the inner diameter 14 of each sleeve 13, which may be a cylindrical inner surface with circular ends, is used to guide a tool for the percutaneous preparation of the bone 3, for the subsequent fitting of one of the transverse fixing screws in the corresponding apertures 2₁, 2₂, 2₃ or 2₄ of the nail 2. The centerline of this sleeve 13 extends along the corresponding axis Z-Z and the body 11 ensures that it is aligned coaxially with one of the fixation apertures 2₁ to 2₄. In FIGS. 1 and 2, the hole 14 defined by sleeve 13 depicted is associated with passage 24.

When using the inner diameter 14 of one of the sleeves 13 in order, for example, to guide a drill bit, the drill bit will act percutaneously to pierce the wall 3₁ of the bone 3 transversely from the outer face of the wall 3₁ to the medullary canal 3₂, and the resulting hole in the bone will be aligned with one of the passages 2₁ to 2₄. In practice, the shape of the body 1 1, and more particularly of its parts 11₁, 11₂, 11₃ and 11₄ may include any shape for which the alignment device 10 is capable of positioning the guide sleeves 13 extracutaneously relative to the nail 2 when the nail 2 is housed in the medullary canal 3₂ of the bone 3.

The instrumentation 1 also includes a system 20 for the percutaneous injection of a substance 5, according to embodiments of the present invention. The substance 5 may be a liquid or a fluid containing biochemical substances such as, for example, bone growth factors, according to embodiments of the present invention. According to other embodiments of the present invention, the substance 5 may be a viscous solution configured to give mechanical reinforcement to the fracture such as, for example, bone cement. According to some embodiments of the present invention, biochemical substances other than or in addition to growth factors may be contained in the substance 5.

As seen in FIG. 2, the injector system 20 includes a generally tubular injector body 21. The cross section of this body 21 is externally fitted to the internal cross section of the sleeve 13, according to embodiments of the present invention. The outer face of the body 21 may be substantially cylindrical, with a circular base whose diameter is equal to that of the inner diameter 14 of sleeve 13, according to embodiments of the present invention.

In the configuration shown in FIG. 2, the solution 5 is contained in the internal volume of the distal part of the injector body 21, while the proximal part of this body 21 is equipped internally with a plunger 22 capable of pushing the solution 5 through the distal end part 23 of the injector body 21 and out of the body 21. For this purpose the plunger 22 is mounted so as to slide hermetically inside the body 21, so that the injector system 21 is somewhat similar to a syringe, although its design and dimensions enable it to be used with the alignment device 10, according to embodiments of the present invention.

The overall shape of the end part 23 of the injector body 21 is a tube inscribed within the geometrical projection of the body 21, with an outside transverse section that is both coaxial with the body 21 and smaller than the outside transverse section of the body 21, according to embodiments of the present invention. At its longitudinal end connected to the body 21, the internal volume of the part 23 opens unobstructedly into that of the body 21, according to embodiments of the present invention. The free end of the part 23 opens unobstructedly to the exterior through an outflow orifice 24 coaxial with the body 21. The end part 23 may have other outflow orifices, such as, for example, outflow orifices 25, connecting the internal volume of the end part 23 to the exterior in a direction substantially radial with respect to the central axis of the injector body 21, according to embodiments of the present invention.

Other features of the instrumentation 1 are also readily apparent from an example of the use of this instrumentation for treating a fracture F (FIG. 2) of the bone 3. A first step is for the surgeon to install the nail 2 in the medullary canal 3₂ of the bone 3 by inserting it into the canal 3₂ via one of the longitudinal ends of the bone 3. The position of the nail thus placed inside the medullary canal 3₂ is determined by the alignment device 10 connected to the nail by the screw 4, as described above. Insertion of the nail 2 into the medullary canal 3₂ is comparatively uninvasive because it is performed only through the soft tissue lying in the longitudinal projection of this medullary canal 3₂, according to embodiments of the present invention.

For a second step, the surgeon uses the alignment device 10 to prepare the bone 3 percutaneously. In particular, as also explained above, the surgeon inserts into the sleeve 13 one or, in succession, multiple percutaneous preparation tools to enable the subsequent fitting of a transverse fixing screw in the passage 2₄ between the bone 3 and the nail 2. For example, the surgeon may insert a drill bit into the sleeve 13 until it penetrates the wall 3₁ and thus reaches the medullary canal 3₂, resulting in a drilled hole 3₃ in the bone.

For a third step, the surgeon uses the injector system 20 as illustrated in FIG. 2. More precisely, the surgeon inserts into the sleeve 13 the injector body 21 containing the solution 5, until the end part 23 of the body 21 reaches the hole 3₃, through which the end part 23 advances until it reaches the medullary canal 3₂. The end part 23 of body 21 may be referred to as an injection nozzle 23. The lengthwise dimension of the nozzle 23 is such that its free end advances into the entrance of the passage 2₄, without actually extending into the passage 2₄. In this configuration, when the surgeon pushes the plunger 22 towards the nozzle 23, by acting on that end of the plunger which is located at the proximal end of the body 21 as indicated by arrow F₁ in FIG. 2, the solution 5 is correspondingly driven through the nozzle 23, as indicated by the arrow F₂. The solution 5 is discharged from the nozzle 23 through both the orifice 24 and the orifices 25, as indicated by the respective arrows F₃ and F₄, according to embodiments of the present invention. The solution 5 passing through the orifice 24 then spreads into the passage 2₄ and can pass out of the lateral side of the nail 2 furthest from the injector system 20, where the solution 5 is spread out through the orifices 25. As a result, the solution 5 spreads into the medullary canal 3₂, all around the nail 2, and reaches the fracture site F in the immediate vicinity of the passage 2₄ in a precise and reliable manner, according to embodiments of the present invention.

According to alternative embodiments of the present invention, the nozzle 23 of the injector body 21 may have other longitudinal dimensions. Thus, if the nozzle 23 is shorter than shown in FIG. 2, its free end does not engage the passage 24, and when the plunger 22 is pushed, the solution 5 spreads out essentially along the lateral side of the nail 2 nearest the injector system 20. Such embodiments may be particularly suited for treatment of a fracture situated mainly on the same lateral side of the bone 3. According to other alternative embodiments of the present invention, the longitudinal dimension of the nozzle 23 is longer than that shown in FIG. 2, in which case the free end part of nozzle 23 extends all or part of the way into the passage 2₄, optionally with the free end of the part 23 projecting out from the outer face 2A of the nail 2. The solution 5 may then be injected essentially, or even exclusively, next to that lateral side of the nail 2 which is furthest from the injector system 20, for example in the treatment of a fracture F located more on the far lateral side of the bone 3. In such cases, the outside transverse section of the nozzle 23 may be smaller or at least slightly smaller than the inside transverse section of the passage 2₄.

In a fourth step, the surgeon removes the injector system 20 by withdrawing the injector body 21 from the sleeve 13. This sleeve is then used to guide the placement, into the passage 2₄, of a transverse fixation screw between the nail 2 and the bone 3, according to embodiments of the present invention.

If appropriate, the injector system 20 or similar systems may be used, concomitantly or in succession, in conjunction with one or more sleeves 13 of the alignment device 10 other than the single sleeve 13 shown in FIGS. 1 and 2. Depending on which sleeve 13 is actually used to guide, with its inner diameter 14, the injector body 21, the solution 5 is injected at or even into the corresponding one of the passages 2₁ to 2₄ of the nail 2. The one or more particular alignment sleeves 13 used to inject the solution 5 may thus be chosen by the surgeon to result in desired placement of the solution 5 and for desired or optimal delivery to the fracture site.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A system for injection in osteosynthesis nail installation, the system comprising:
an osteosynthesis nail configured for placement within a canal of a bone, the osteosynthesis nail comprising a fixation aperture;
an alignment device coupled with the osteosynthesis nail, the alignment device comprising an alignment hole having a longitudinal axis passing through the fixation aperture; and
an injector configured for insertion into the alignment hole and into communication with the canal, the injector comprising an injector body with an injection nozzle and a plunger configured to push a substance from within the injector body through the injection nozzle.

2. The system of claim 1, wherein the alignment hole is an alignment sleeve.

3. The system of claim 1, wherein the substance is a fluid containing bone growth factors.

4. The system of claim 1, wherein the substance is bone cement.

5. The system of claim 1, wherein the injection nozzle fits within the fixation aperture.

6. The system of claim 2, wherein the injection nozzle comprises an orifice through which the longitudinal axis of the alignment sleeve passes when the injector is inserted in the alignment sleeve.

7. The system of claim 6, wherein the orifice is a first orifice, and wherein the injection nozzle further comprises one or more radial orifices apart from the first orifice.

8. The system of claim 1, wherein the osteosynthesis nail is a nail selected from the group consisting of a humeral nail, a femoral nail, and a tibial nail.

9. A method for introducing a substance near a fracture of a bone, the method comprising:
installing an osteosynthesis nail into a canal of the bone, the osteosynthesis nail comprising a fixation aperture;
coupling an alignment device with the osteosynthesis nail, the alignment device comprising an alignment sleeve having a longitudinal axis passing through the fixation aperture;
drilling a hole through a wall of the bone and into the canal using the alignment sleeve; and
inserting an injector through the alignment sleeve.

10. The method of claim 9, the injector having an injector body with an injection nozzle and a plunger, the method further comprising:
moving the plunger to push the substance from within the injector body through the injection nozzle and into the canal.

11. The method of claim 9, further comprising:
installing a fixation screw through the alignment sleeve and into the fixation aperture.

12. The method of claim 9, wherein the injector comprises an injection nozzle, and wherein inserting the injector through the alignment sleeve comprises inserting an end of the injection nozzle until the end is situated at an opening in an outer surface of the bone.

13. The method of claim 9, wherein the injector comprises an injection nozzle, and wherein inserting the injector through the alignment sleeve comprises inserting an end of the injection nozzle until the end is situated at an opening in an inner surface of the bone.

14. The method of claim 9, wherein the injector comprises an injection nozzle, and wherein inserting the injector through the alignment sleeve comprises inserting an end of the injection nozzle until the end is situated at the fixation aperture.

15. The method of claim 9, wherein the injector comprises an injection nozzle, and wherein inserting the injector through the alignment sleeve comprises inserting an end of the injection nozzle until the end is situated in the fixation aperture.

16. The method of claim 9, wherein the injector comprises an injection nozzle, and wherein inserting the injector through the alignment sleeve comprises inserting an end of the injection nozzle entirely through the fixation aperture.
